(19) United States
(12) Patent Application Publication
(10) Pub. No.: US 2022/0298080 A1
(43) Pub. Date: Sep. 22, 2022

(54) POLYCRYSTALLINE CERAMIC SOLID, DIELECTRIC ELECTRODE COMPRISING THE SOLID, DEVICE COMPRISING THE ELECTRODE AND METHOD OF PRODUCTION

(71) Applicant: TDK Electronic AG, München (DE)

(72) Inventors: Manfred Schweinzger, Bad Schwanberg (AT); Andreas Pentscher-Stani, Leibnitz (AT)

(21) Appl. No.: 17/641,391

(22) PCT Filed: Sep. 30, 2020
(86) PCT No.: PCT/EP2020/077394

§ 371 Date: Mar. 8, 2022

(30) Foreign Application Priority Data
Sep. 30, 2019 (DE) 10 2019 126 346.8

(51) Int. Cl. C04B 35/468; C04B 35/64; A61N 1/06

(52) U.S. Cl. CPC C04B 35/4682; C04B 35/64; A61N 1/06; C04B 2235/3229; C04B 2235/3244; C04B 2235/3262; C04B 2235/786; C04B 2235/79

(57) ABSTRACT

A polycrystalline dielectric solid body has a main phase of the general formula Ba_{0.995}(Ti_{0.85}Zr_{0.15})O₃ and is co-doped with manganese and a rare earth element. The solid body can be used as a dielectric electrode in a method for treating tumors with alternating electric fields.

Polycrystalline ceramic solid body, dielectric electrode comprising the solid body, device comprising the electrode, and method of making the electrode

The invention relates to a polycrystalline ceramic solid body suitable as an electrode material for applying alternating fields to the human or animal body. Further, the invention relates to an electrode comprising the ceramic solid body and a device comprising said electrode, the device being suitable for applying alternating fields to the human or animal body. Finally, the invention relates to a process of producing a ceramic solid body and an electrode comprising the solid body.

From the prior art, processes are known which inhibit cell division in organisms by applying electric fields. This principle can be used for the treatment of a number of tumor types by inhibiting the rapid and uncontrolled cell division of tumor cells by applying high-frequency alternating electric fields. Corresponding procedures have been approved by the US. Food & Drug Administration (FDA). The high-frequency alternating electric fields used to combat tumor cells are also known as “tumor treating fields” (TTF). They are transmitted to the patient by means of ceramic electrodes placed around the body region affected by the tumor. Selectivity for different cell types can be achieved by choosing appropriate frequencies. This mitigates the side effects of the therapy. Examples of processes and devices for destroying uncontrollably dividing cells, can be found, for example, in US patent application US 2003/0150372 A1 and patent U.S. Pat. No. 7,016,725 B2.

A special role in the processes described is played by ceramic electrodes, which are used to transmit the high-frequency alternating electric fields to the organism to be treated. There is a great need for new materials suitable for this purpose.

From the Austrian utility model GM50248/2016, a lead-containing polycrystalline ceramic solid body is known for such an application, which has a main phase of the following general formula:

(1−y)Pbₐ(Mg_{b}Nb_{c})O₃₋ₑ+yPbₐTi_{d}O₃

One object of the present invention is to provide new materials that can be used as electrodes for efficient transmission of high-frequency alternating electric fields to the human or animal body. In particular, a lead-free material with suitable properties meeting the specifications is sought.

This task is solved by a material according to claim 1.

A polycrystalline dielectric solid body is proposed which has a main phase with a perovskite structure and the general formula Ba₁₋ₐ(Ti_{1-b}Zr_{b})O₃ and is co-doped with manganese and a rare earth element. Here, a and b are smaller than 1 and greater than zero. A preferred doping reaches a concentration of 0.1 at % at most.

According to a first embodiment, the invention relates to a polycrystalline ceramic solid body comprising a main phase having an ABO₃ perovskite structure comprising a composition of the following general formula:

Baₘ(TiₙZrₚ)0₃

and a dopant of the general formula:

MnₓRE_{z}

wherein RE represents one or more rare earth elements wherein the following applies for the coefficients:
m=0.95 to 1.05
n=0.8 to 0.9
p=0.1 to 0.2
x=0.0005-0.01
z=0.001-0.050
   wherein the following applies:
m<(n+p)
   whereby the B components are in excess in the ABO₃ lattice.

In particular, the ratio x/z of the proportions of components Mn and RE of the doping is set in the range 1:2 to 1:10.

In this context, a polycrystalline solid body is understood to be a crystalline solid body that comprises crystallites, which are also referred to as particles or grains in the following. The crystallites are separated from each other by grain boundaries. Accordingly the solid body contains grains which contain or consist of the material of the main phase. A solid body is in particular sintered. In particular, the grains have a diameter in the range of several μm.

In addition to the main phase, the proposed lead-free solid body may also contain secondary phases. The solid body can be manufactured such that secondary phases are undetectable in the particles containing the main phase. The secondary phases have single or multiple components contained in the main phase in different composition and different structure from that of the main phase or undefined structure.

In particular, the proposed lead-free solid body has at least a first secondary phase that is rich in the component RE and a second secondary phase that is rich in Ti, which are predominantly or completely arranged in multi junction grain boundaries between the particles of the main phase.

Since secondary phases differ from the main phase in their elemental composition, it is possible to quantify the area fraction of the secondary phase relative to a sectional area through the solid body by means of element distribution images. Such elemental distribution images can be obtained using SEM-EDX measurements (SEM stands for scanning electron microscopy; EDX stands for energy dispersive X-ray spectroscopy).

It is a key feature of the proposed solid body that it has only a small proportion of secondary phases, but this must not assume zero, since the secondary phases also help to determine the particularly advantageous properties of the solid body. Thus, in any given section through the solid body, the area fraction of all secondary phases added together, based on the sectional area through the solid body, is less than or equal to 1% and preferably less than 0.3%.

The solid body exhibits properties that meet the specification of the desired application. In particular, it exhibits an exceptionally high dielectric constant c of more than 40000 measured at 35° C. It is found that the c of the solid body exhibits its maximum in a temperature range between 30 and 42° C. A high dielectric constant in this range is particularly favorable for use in ceramic electrodes on the body of patients, since particularly high capacitances can thus be achieved at body temperature.

The maximum capacitance can be set, for example, by the ratio of the B component (Ti, Zr) to the A component.

The high capacitance makes the solid body very suitable for use as an electrode for the above-mentioned process, in which cell division in organisms can be inhibited by applying electric fields via the electrodes. These high-frequency alternating electric fields used to combat tumor cells are also known as “tumor treating fields” (TTF).

Good properties are exhibited by a solid body in which the at least one rare earth element RE used for doping is selected from Pr, Dy, Ce and Y or comprises a combination thereof.

Advantageously, the solid body is one in which the main phase is in the form of particles having uniform orientation within a particle, and in which the particles have a mean grain size d₅₀ of 10 to 30 μm, measured as a number median value by static image analysis. In a measurement method addressing the determination of or distribution of the grain sizes, the SEM/EBSD (Electron Backscattering Diffraction) contrast on a cut surface of the solid body can be used.

In addition to the secondary phases, the solid body may also have porosity. Presumably as a result of the high proportion of grains with the pure and uniform main phase, the porosity does not occur in open pores, i.e. the vast majority of the pores are completely embedded in the solid body and are not in contact with a medium (atmosphere or an application-related medium such as a gel). Accordingly, the maximum absorption of moisture is also low.

The solid body can have a closed porosity between 0.1 and 1.1 volume %, mostly less than 0.5%.

The polycrystalline ceramic solid body is characterized by high mechanical stability. Components such as electrodes formed from this material are therefore robust and durable.

In addition, the material proposed for the solid body has a high breakdown voltage. Such a high breakdown voltage is important for safe application as electrode material on the patient, as it helps protect the patient from high currents through the body and resulting damage.

A preferred application of the solid body is as a dielectric electrode in a device for TTF therapy. For this purpose, the solid body is formed in the shape of a relatively thin disk and provided with a metallic coating for electrical contacting.

A device for the treatment of an animal or human body with TTF preferably comprises at least two such electrodes, which can have a diameter of 0.2 to 2.5 cm, depending on the application. Such electrodes are then placed directly on the body surface in the area of the degenerated cells or glioblastomas to be treated and coupled to the body via a mediating medium such as a gel and attached there.

Treatment can then extend over several weeks or months, during which the electrodes are exposed to an alternating electric field of a frequency of more than one hundred kilohertz. During this process, the polycrystalline solid body has a sufficiently high dielectric strength so that there is no flashover and thus no damage to the treated body or body part. The high dielectric strength can also be maintained during continuous operation under normal environmental conditions. In one embodiment, for example, it was shown that an electrode according to the invention still exhibits a breakdown voltage of 4.8 kV even after 24 hours of storage in a 0.9% saline solution, which is significantly higher than the voltages used during operation of the device. Furthermore, at a typical layer thickness of about 1 mm, the solid body of the electrode still exhibits an insulation resistance of, for example, 6 GOhm after the 24-hour saline storage.

In a process of producing a ceramic solid body according to the invention, starting materials are provided containing the components Ba, Ti, Zr, Mn and RE, in a proportion corresponding to the composition of the main phase and the doping. In steps known per se, the starting materials are ground, homogeneously mixed, calcined under air and, after optional further steps, converted into a green body. Preferably, the calcinate is pressed dry to form the green body. The green body is then sintered to a solid body under an oxidizing atmosphere, e.g. under air, at a sintering temperature between 1400 and 1500° C. The sintering temperature should be maintained as accurately as possible, since it has a significant effect on the electrical properties of the solid body.

To produce an electrode, the polycrystalline ceramic solid body is provided with a metal layer of a thickness of, for example, 1 to 25 μm in a subsequent step.

Electrical contact can be made by applying a paste to the sintered solid body and then baking it, the baking preferably being carried out at a temperature of 680 to 760° C. The sintered solid body is then coated with a metal layer of a thickness of, for example, 1 to 25 μm.

However, it is also possible to apply the contacting by means of a thin-film process or suitable other processes.

In the following, the invention will be explained in more detail with reference to exemplary embodiments and the accompanying figures. Unless they are measurement results, the figures are schematic and may not be true to scale for better understanding.

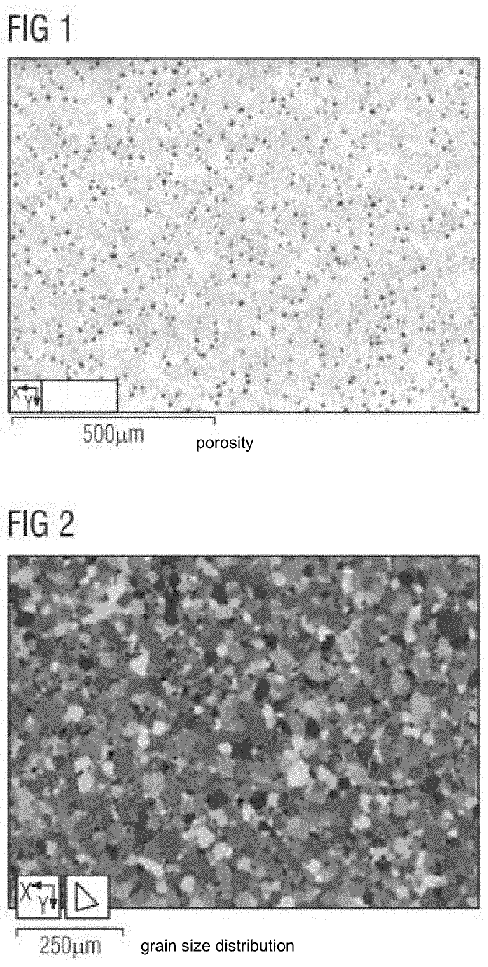

FIG. 1 shows an EBSD image for the determination of the porosity of the solid body

FIG. 2 shows the SEM/EBSD contrast of a solid body according to an embodiment example for the determination of the grain size distribution

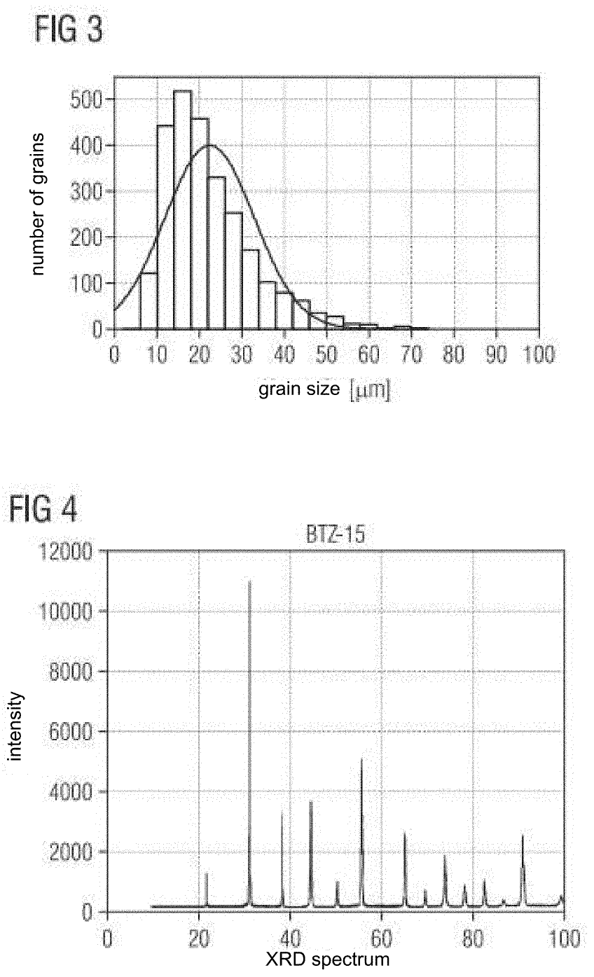

FIG. 3 shows the grain size distribution of the solid body as a histogram

FIG. 4 shows an XRD diagram of the solid body

FIG. 5 shows the temperature dependence of the capacitance of the solid body compared to a know (lead-containing) approach

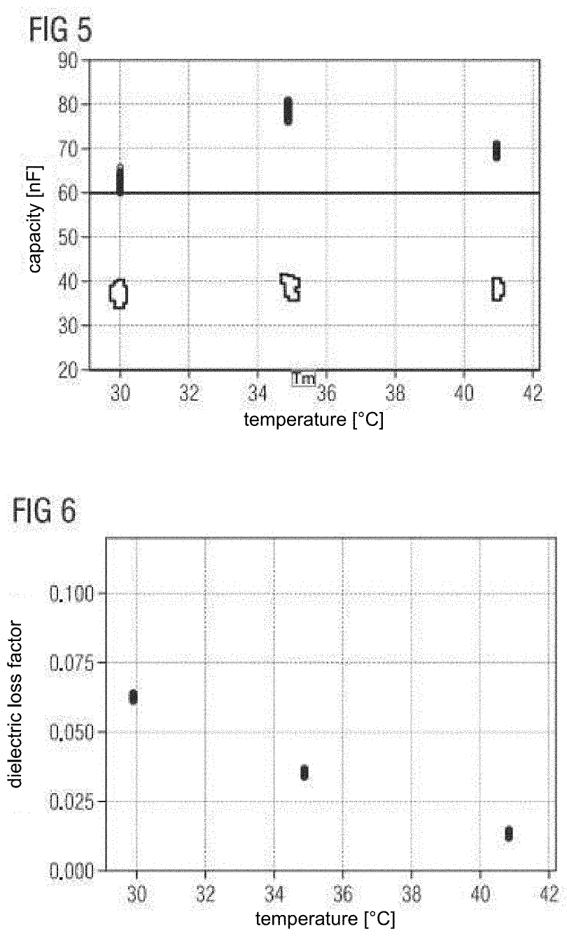

FIG. 6 shows the temperature dependence of the dissipation factor as an example the solid dielectric losses of the solid body

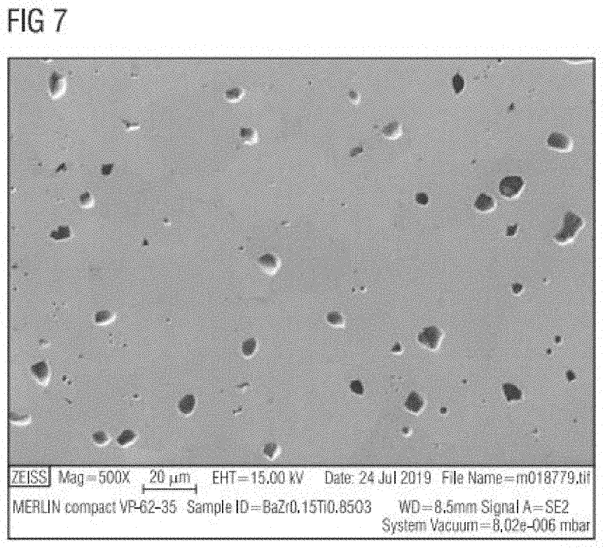

FIG. 7 shows a SEM image of the solid body

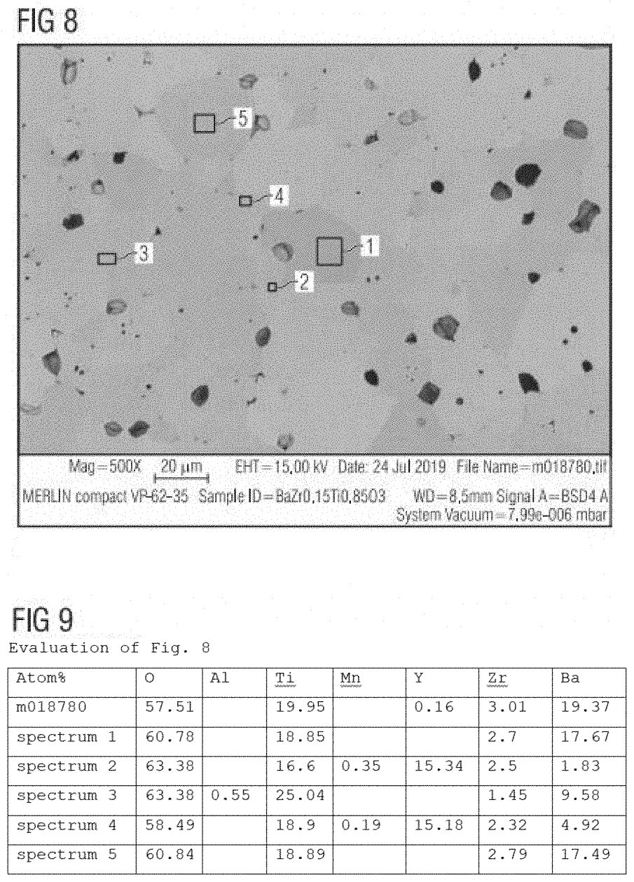

FIG. 8 shows an SEM image of the solid body with BSE contrast.

FIG. 9 shows in a table the local composition of selected areas of the SEM image of FIG. 8

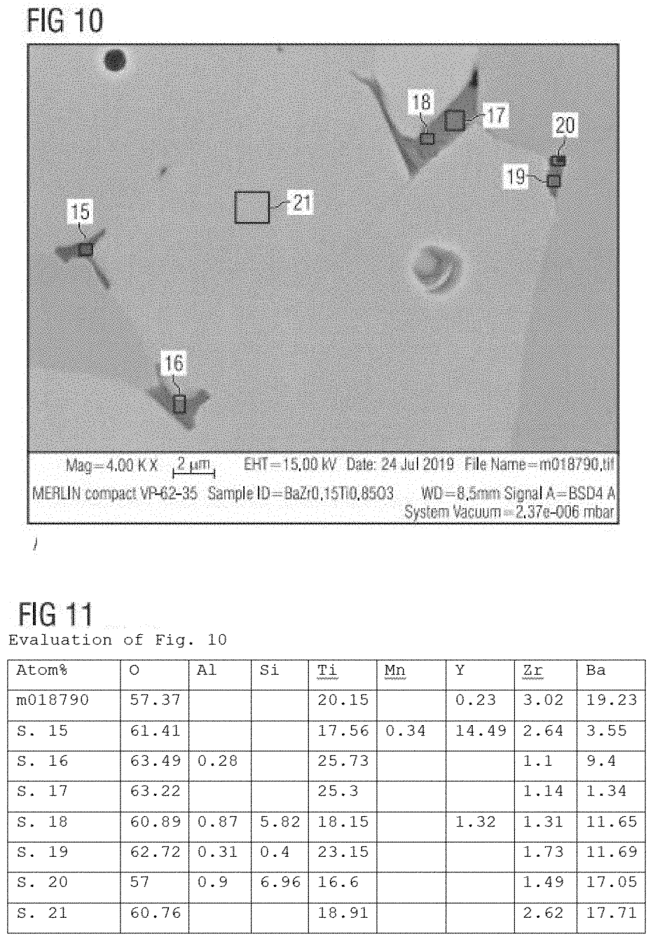

FIG. 10 shows another SEM image of the solid body with BSE contrast

FIG. 11 shows in a table the local composition of selected areas of the SEM image of FIG. 10 of the solid body

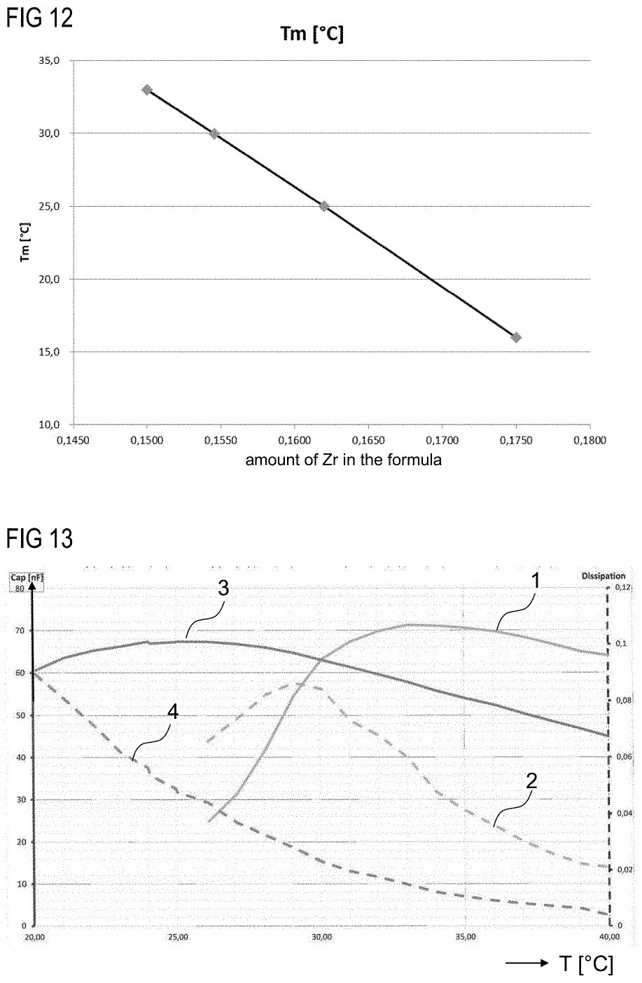

FIG. 12 shows the dependence of the temperature Tm of the capacitance maximum on the Zr fraction in the solid body

FIG. 13 shows the dependence of capacitance and dielectric loss factor on temperature for two solid bodies of selected composition.

### EXEMPLARY EMBODIMENTS

A first specific example of a polycrystalline solid body with the above properties has the following composition:

Ba_{0.995}(Ti_{0.850}Zr_{0.150})O₃+0.002at % Mn and +0.01at % Y

Starting components for the solid body with the above composition are provided in a ratio corresponding to the formula and processed into a raw body by common ceramic processes such as grinding, calcining, spray drying, pressing, and so on. The raw body is then sintered at a temperature of about 1400° C. to 1500° C., for example at 1450° C.

A polycrystalline solid body is obtained which has a porosity of less than 5%. Advantageously, the solid body can also have a porosity of about 1 volume % or less.

The porosity of the exemplary embodiment is determined from a polished cross section of the solid body by SEM analysis using EBSD. EBSD stands for “electron back scatter detection.” This is the detection of electron diffraction. For each point on the examined surface a diffraction pattern is examined, from which information on the crystal orientation in this point is extracted.

Within a grain, each point has the same crystal orientation, as is always the case in a poly- or microcrystalline structure of a ceramic. Directly adjacent grains have different crystal orientations with a high statistical probability. Therefore, grain boundaries can be observed or determined with this method.

By means of image analysis, it is now possible to perform a quantitative analysis of the grain size distribution. With this method, crystal regions (ground grains) associated with the main phase are detected. The parts of the surface that do not correspond to the main phase Ba_{0.995}(Ti_{0.850}Zr_{0.150})O₃ detected by means of EBSD are evaluated as pores. The amount of minor phases is so small that it is below the detection limit of the mentioned method, so the minor phases are therefore not detected in the method. Accordingly, the area fractions not corresponding to the main phase (zero solution) can be evaluated as porosity.

FIG. 1 shows the EBSD image of the polished cross section of the solid body according to the first exemplary embodiment. The dark dots correspond to the pores and occupy an area fraction of about 3% on the cross-sectional surface. In the following, the pores are also referred to as zero solutions, insofar as it is referred to the optical analysis of a polished cross section of the solid body. For the exemplary embodiments, the phase distribution determined by counting is as follows:

| phase name | phase fraction | phase count |
|---|---|---|
| BaZr_{0.15}Ti_{0.85}O₃ | 96.97% | 236851 |
| zero solution | 3.03% | 7389 |

The solid body has a density of 5.6-5.8 g/cm³.

The grain size determined by SEM/EBSD contrast is typically 20 μm. FIG. 2 shows the SEM/EBSD contrast of the solid body according to the embodiment example. In the image, the different grains can be easily recognized and evaluated by contrast imaging. In the embodiment example, a more accurate value of 21.9 μm+/−8.4 μm is obtained.

FIG. 3 shows the grain size distribution of the solid body determined from the image in FIG. 2 as a histogram. It can be seen that most of the grain sizes are between 10 and 30 μm. The histogram shows that the solid body has a relatively narrow grain size distribution.

Furthermore, the phase composition and its crystal structure is determined by XRD analysis and energy dispersive X-ray, respectively. For this purpose, the solid body is embedded in resin, ground and polished with silica gel. To avoid charging, the sample is vapor coated with a thin conductive carbon layer.

XRD analysis reveals a crystal structure that is 100% tetragonal with a c/a ratio of 1.001-1.003. The percentage of secondary phases is less than 0.5%, which is below the detection limit of the XRD analysis method. From the fact that no secondary phases can be detected, the embodiment must have a phase purity of more than 99.5%.

FIG. 7 shows an SEM image of the solid body resolved by secondary electrons, showing the topography contrast of the examined surface of the polished cross section of the solid body.

FIG. 8 shows an SEM image of the solid body resolved by back scattered electron (BSE) contrast. From the energy of the scattered electrons, elements can be identified or resolved according to their nuclear charge number.

Elements with higher nuclear charge numbers can be identified in the image by their higher brightness. The image shows that, in addition to the assignable main phase, other secondary phases are present in the solid body. The distribution of the secondary phases, which can be taken from the image, shows that they occur or form exclusively at grain boundaries or multi junction grain boundaries of main phase grains.

Surface areas of the cross-section are now examined for their exact composition. In FIG. 8, these areas are highlighted with a frame and assigned a number. By resolving the energy of the backscattered electrons, element distribution images can be generated and the exact element content of the examined surface areas can be determined.

The table in FIG. 9 shows the element contents of the surface areas investigated. It can be seen that areas 2 and 4 are rich in Y and can therefore be assigned to a Y segregation phase. Other elements are also detected, but this is essentially because the electron beam is examining a larger volume than corresponds to the extent of this phase. This phase consists essentially of Y₂O₃. Area 3 has a phase rich in the element Ba, which can be assigned to a secondary phase.

FIG. 10 also shows an SEM image of the solid body which has been resolved according to a BSE contrast. It shows a different section of the investigated surface. Here, too, different surface areas are highlighted with a frame and assigned a number. The table in FIG. 11 shows the element contents of the surface areas investigated.

Here it can be seen that areas 16, 17 and 19 exhibit a phase rich in the element Ba, essentially comprising BaTi₂O₅. Areas 15 and 18 have a phase rich in the element Y, but in area 15 this is overlaid by a phase containing Ba(Ti/Zr)O₅, while in area 18 this is overlaid by the main phase.

The total area of the cross section examined was 114 μm×86 μm=9804 μm². The measured areas of the Y-rich and Ti rich phases have an average area of 2 μm² each. In the section examined, 4 areas of Y-rich phases corresponding to approximately 8 μm² total area and 8 areas of Ti rich phase corresponding to approximately 16 μm² were found.

This results in an approximate area fraction with predominantly secondary and minority phases as follows:

Y-rich phase: −0.08% (Y₂O₃)

Ti-rich phase: −0.16% (BaTi₂O₅)

This corresponds to a volume fraction of

Y-rich phase: 0.0023 volume % (Y₂O₃)

Ba-rich phase: 0.0659 volume % (BaTi₂O₅)

It is assumed that the following effects can be attributed to the secondary phases found, which together account for the advantageous properties of the solid body. BaTi₂O₅ has a melting point of 1.320° C., which is lower than the sintering temperature of the main phase used. Thus, the BaTi₂O₅ phase appears to form an intrinsic sintering aid in the sintering process of the solid body.

The Y₂O₃ enrichments at the grain boundaries can act as donor dopants and have a positive effect on the insulation resistance. In this way, the charge clouds can be bound in a locally stable manner in the doped solid boy of the main phase, and are then no longer mobile, ensuring that no mobile charge clouds undesirably increase the conductivity of the solid body.

Solid bodies with a similar composition of starting components are already known for other applications, but these are multilayer ceramic devices such as a multilayer capacitor known, for example, from U.S. Pat. No. 5,014,158 A. These devices have metallic inner electrodes that limit the maximum sintering temperature to the melting point of the inner electrode metal. For example, ceramic bodies with nickel inner electrodes have so far been sintered exclusively under reducing conditions at well below 1500° C. so as not to damage the Ni inner electrodes.

A solid body according to the invention, on the other hand, has no internal electrodes and is sintered at a significantly higher temperature and under air, i.e. under an oxidizing atmosphere. Due to the aggregation processes described above, which occur only at higher sintering temperature (as used in all embodiments) and which have effects on the electrical properties of the solid body, the invention provides a solid body with improved electrical properties. These properties could not be observed in a known component such as the aforementioned multilayer capacitor due to the lower sintering temperatures used so far and the mandatory reducing sintering atmosphere.

To determine the electrical properties of the new solid body, solid bodies are produced in a target geometry as required or particularly suitable for use as an electrode for a TTF therapy application. In particular, such a geometry is a disc with a hole with an outer diameter of about 19 mm, an inner diameter of about 3 mm and a thickness of about 1 mm. These disks are provided with a metallization of e.g. Ag of approx. 10 μm thickness. In particular, the temperature dependence of the capacitance, the dielectric constant, the dielectric loss factor, the breakdown voltage after 24 hours of storage in approx. 1% aqueous saline solution and the insulation resistance also after storage in saline solution are determined.

The capacitance measurement is performed at an applied AC voltage of a frequency of 200 kHz.

Storage in saline solution is intended to simulate conditions that may exist after prolonged contact of electrodes made from the solid state for a TTF procedure with the skin of patients in the vicinity of the electrodes. Passing this test accordingly promises a long possible operating life of corresponding electrodes directly on the human body. Also, the breakdown voltage determined at the disc with a hole is sufficiently high at about 4.8 kV and the insulation resistance of a 1 mm thick layer of the solid body after storage in 1% saline solution still reaches 6 GOhm.

FIG. 5 shows in the upper part of the diagram the temperature dependence of the capacitance determined on the disc of the first embodiment. It can be seen that the capacitance has a maximum of approximately 78 nF at a temperature Tm of approximately 35. This is particularly advantageous in that a high capacitance is desired for the TTF application and this is reached exactly in the range of the body temperature of a human being.

In the lower part of the diagram, the temperature dependence of the capacitance of known lead-containing ceramics, as they have been used so far for a TTF process, is shown for comparison. Although lead-containing ceramics also show a maximum at a temperature of about 35° C., these capacitance values reach at most about half the capacitance value of the new polycrystalline solid body.

The dielectric losses decrease with increasing temperature and reach a sufficiently low value of about 6% at 35° C. FIG. 6 shows the temperature dependence of the dielectric losses of the solid state according to the invention.

At the same AC voltage and a temperature of 35° C., a dielectric constant ε of more than 40.00 is determined. This ε by far exceeds the ε of known lead-containing TTF electrode materials, which is about 25.00. A high ε is advantageous for the application of the solid body as a dielectric electrode material. In sum, the superiority and especially the excellent properties of the new material can be obtained with the proposed co-doping with Mn and a rare earth element.

In further experiments, additional dielectric solid bodies were prepared by the same method as in the first embodiment. Here, the composition was varied exclusively with respect to the Zr/Ti ratio, and in all the experiments the Zr/Ti ratio was within the specified limits. The temperature dependence of the capacitance maximum was determined for several of these solid bodies thus obtained.

It was found that the maximum capacitance is obtained with decreasing Zr portion at lower temperatures.

The following table gives the variation of the temperature Tm of the capacitance maximum for different Zr portions:

| Zr | Ti | Tm [° C.] | Zr portion |
|---|---|---|---|
| 0.1500 | 0.8500 | 33.0 | 0.1500 |
| 0.1546 | 0.8454 | 30.0 | 0.1546 |
| 0.1620 | 0.8380 | 25.0 | 0.1620 |
| 0.1750 | 0.8250 | 16.0 | 0.1750 |

FIG. 12 shows the nearly linear dependence of the temperature Tm of the capacitance maximum on the Zr portion in the solid body.

This strong dependence can be used to optimize such dielectric solid bodies of high capacitance for different application temperatures.

FIG. 13 shows the dependence of the capacitance Cap and the dielectric dissipation factor on the temperature for two solid bodies of selected composition.

Curve 1 shows the capacitance profile of a solid body with a composition according to the first exemplary embodiment with a Zr:Ti ratio of 0.150:0.850, while curve 2 shows the profile of the dissipation factor of the same solid body versus temperature.

Curve 3 shows the capacitance profile of a solid body according to a further exemplary embodiment with a Zr:Ti ratio of 0.162:0.838, while curve 4 gives the profile of the dissipation factor of the same solid body versus temperature.

Both solid bodies are completely identical in composition except for the different Zr:Ti ratio. According to curve 3, the capacitance maximum of the second (further) solid body occurs at a significantly lower temperature than that of the first Solid body according to curve 1. The difference here is about 10°.

1. A polycrystalline, ceramic solid body
comprising a main phase obtainable by sintering and having an ABO₃ perovskite structure and a composition of the following general formula:
Baₘ(TiₙZrₚ)O₃
and a doping of the composition
MnₓRE_{z}
wherein RE represents one or more rare earth elements,
wherein the following applies for the coefficients:
m=0.95 to 1.05
n=0.8 to 0.9
p=0.1 to 0.2
x=0.0005 to 0.01
z=0.001 to 0.050
wherein the following applies:
m<(n+p)
whereby the B components of the ABO₃ lattice are present in excess.

2. The solid body according to claim 1, wherein RE is selected from Pr, Dy, Ce, Y and a combination thereof.

3. The solid body according to claim 1, wherein the ratio of the proportions of components Mn and RE of the doping is set in the range of 1:2 to 1:10.

4. The solid body according to claim 1, wherein the main phase is in the form of particles having uniform orientation within one particle, in which the particles have a mean particle size d₅₀ of 10 to 30 μm, measured as a number-related median value by static image analysis.

5. The solid body according to claim 1, in which at least a first secondary phase rich in the component RE and a second secondary phase rich in Ti are present, which are predominantly or completely arranged in multi junction grain boundaries between the particles of the main phase.

6. The solid body according to claim 1, which has a closed porosity between 0.1 and 1.1 volume %.

7. The solid body according to claim 1, wherein at any cut through the solid, the area fraction of all secondary phases relative to any cut area through the solid is less than or equal to 1% or less than 0.3%.

8. The solid body according to claim 1, which has a dielectric constant ε determined at 35° C. of ε>40000.

9. The solid body according to claim 1, which has been obtained by sintering at a temperature of 1400 to 1500° C.

10. The solid body according to claim 1, which has been obtained by sintering under air.

11. A dielectric electrode comprising a solid body according to claim 1, which is formed as a ceramic disk with a metallic coating for contacting.

12. A device for applying alternating electric fields to the human or animal body comprising at least one electrode according to claim 1.

13. A process of producing a ceramic solid body according to claim 1,
in which the starting materials comprising Ba, Ti, Zr, Mn and RE are used in a proportion corresponding to the composition of the main phase and the doping,
in which the starting materials are ground and mixed
in which a green body is produced from the starting materials
in which the green body is sintered to form the ceramic solid body.

14. A process of manufacturing an electrode according to claim 11, comprising a method of manufacturing a polycrystalline ceramic solid body according to claim 13 and a subsequent step of providing the solid body with an electrical contact.

15. The process according to claim 14, wherein the electrical contacting is carried out by applying and baking a paste, the baking being carried out at a temperature of 680 to 760° C.

16. The process according to claim 14, wherein the contacting is applied by means of a thin film process.

17. The process according to claim 13, wherein the green body is sintered under air at a sintering temperature between 1400 and 1500° C. to form the solid body.
